(19) United States
(12) Patent Application Publication
(10) Pub. No.: US 2017/0071473 A1
(43) Pub. Date: Mar. 16, 2017

(54) OPTICAL SENSOR FOR A CATHETER

(71) Applicant: KONINKLIJKE PHILIPS N.V., Eindhoven (NL)

(72) Inventors: Manfred MUELLER, Eindhoven (NL); Chaitanya DONGRE, Vaals (NL); Michel Paul Barbara VAN BRUGGEN, Helmond (NL); Ruud VLUTTERS, Eindhoven (NL); Frederik Jan DE BRUIJN, Eindhoven (NL); Waltherus Cornelis Jozef BIERHOFF, Veldhoven (NL); Dirk PETERS, Eindhoven (NL)

(21) Appl. No.: 15/305,707

(22) PCT Filed: Apr. 22, 2015
(86) PCT No.: PCT/EP2015/058733

§ 371 Date: Oct. 21, 2016

(30) Foreign Application Priority Data
Apr. 25, 2014 (EP) 14166049.8
Apr. 25, 2014 (EP) 14166061.3
Oct. 10, 2014 (EP) 14188392.6

(51) Int. Cl. A61B 5/00; A61B 8/12; A61B 1/267; A61B 5/107; A61B 1/05; A61B 1/06

(52) U.S. Cl. CPC A61B 5/0084; A61B 1/05; A61B 1/0615; A61B 1/267; A61B 5/1079; A61B 8/12; A61B 5/4818

(57) ABSTRACT

An optical sensor for operating along a catheter (103), the optical sensor comprising a light pattern generator configured to project at least one light pattern at a radial projection angle with respect to the optical sensor length onto the inner surface of an elongated volume into which the optical sensor is inserted, wherein the light pattern generator comprises an illumination system for providing a first light beam having a component of its direction along the sensor length and a first light redirection element (1007) configured to redirect the light beam to generate the at least one light pattern (1009) at an oblique and/or right angle to the optical sensor length, a second light redirection element (1012) for redirecting a reflected version of the light pattern from the inner surface of the elongated volume to provide a second light beam and- an imaging device (1013) having a field of view (1011) with a central axis substantially parallel to the length of the optical sensor for receiving the second light beam.

### FIELD OF THE INVENTION

This invention relates to optical sensors, and specifically but not limited to upper airway catheter optical sensors for monitoring the shape of the upper airway in sleeping users.

### BACKGROUND OF THE INVENTION

Respiratory disorders during sleep are recognized as a common problem with significant clinical consequences. Obstructive Sleep Apnoea (OSA) causes an intermittent cessation of airflow. When these obstructive episodes occur, an affected person will transiently arouse. As these arousal episodes typically occur 10 to 60 times per night, sleep fragmentation may produce excessive daytime sleepiness. It is known that some patients with OSA experience over 100 transient arousal episodes per hour. OSA may also lead to cardiovascular and pulmonary disease.

Various approaches are known which aim to maintain the airway passage during sleep. Oral appliances aimed at changing the position of the soft palate, jaw or tongue are available, but patient discomfort has limited their use. Continuous Positive Airway Pressure (CPAP) devices are often used as first-line treatments for OSA. These devices use a sealed mask which produces airflow at a slightly elevated pressure and acts to maintain positive air pressure within the airway.

The Obstructive Sleep Apnea (OSA) patient pool eligible for treatments other than Positive Airway Pressure (PAP) is increasing as nowadays more alternatives become available. Unlike PAP, none of these alternatives is ‘one-size-fits-all’ because of the multi-level pathophysiology of OSA. Patient selection is required to ensure optimal clinical outcomes.

Whereas drug induced sleep endoscopy (DISE) is advocated as the best method for patient and therapy selection, many Ear, Nose and Throat (ENT) medical practitioners in Europe and even more in the US do not use DISE because of the associated costs (expensive clinical setting and required staff) and perceived risk (sedation of apnea patients). Furthermore DISE is not considered representative of natural sleep and therefore the collapse patterns observed may not reflect the real pathophysiology of OSA for the specific patient during natural sleep. At present there is no consensus on how to best select OSA treatment alternatives for patients. Pressure catheters try to overcome some of the limitations associated with DISE, but upper airway manometry has shortcomings as well since it only identifies collapse location, but doesn't provide any information regarding the configuration and severity of the upper airway obstructions. Upper airway manometry also provides no visual confirmation in case of an event. Optical catheters and endoscopes have been proposed as a concept to quantify airways. Examples of optical endoscopes can be found from Muller et al “Noncontact three-dimensional laser measuring device for tracheoscopy”, Annals of Otology, Rhino logy and Laryngology, Vol. 111 No. 9 pp. 821-827 (September 2002) and Dorffel et al “A new bronchoscopic method to measure airway size” European Respiratory Journal, Vol. 14 pp. 783-788 (1999), but the scanning approaches described are typically cumbersome in the sense that they require translation of the catheter through the upper airway in order to determine a complete picture of the upper airway. This implies that dynamic information of the upper airway as a whole is lost. Furthermore long-term observation of the upper airway without continuous clinical supervision is difficult. Also the typical optical catheter has an optical sensor which at best has a field of view which significantly limits the ability to monitor a substantial cross sectional area and therefore can miss or miss diagnose the type of event occurring in the airway as the optical catheter is ‘directed’ in a direction which fails to capture the event or capture the event significantly well.

### SUMMARY OF THE INVENTION

The invention is defined by the claims.

According to the invention, there is provided an optical sensor for operating along a catheter, the optical sensor having a sensor length along a direction parallel to the catheter direction, the optical sensor comprising:

a light pattern generator configured to project at least one light pattern at a radial projection angle with respect to the optical sensor length onto the inner surface of an elongated volume into which the optical sensor is inserted, wherein the light pattern generator comprises an illumination system for providing a first light beam having a component of its direction along the sensor length and a first light redirection element configured to reflect the light beam to generate the at least one light pattern at an oblique and/or right angle to the optical sensor length;

a second light redirection element for redirecting a reflected version of the light pattern from the inner surface of the elongated volume to provide a second light beam; and

an imaging device having a field of view with a central axis substantially parallel to the length of the optical sensor for receiving the second light beam.

The optical sensor is for example an elongate structure along the catheter with which it is to be used, so that the elongate axis direction corresponds to the catheter length direction. The imaging device field of view has a central axis substantially parallel to the length direction of the optical sensor, and the light pattern generator is configured to project light radially with respect to the length direction of the optical sensor.

The optical sensor may be at the tip of the catheter, or it may be somewhere else along the catheter length, so that the catheter continues on either side of the sensor. The first light beam may be generally aligned along the sensor length, so that the first light redirecting element converts an axial direction to a radial direction. The second light beam may also be generally aligned along the sensor length, for example converging at the imaging device. The second light redirecting element thus convers from a radial direction to an axial direction.

This arrangement in some embodiments thus enables a catheter comprising such a sensor within and along the catheter to make measurements while the catheter is static or stationary within the volume (such as an upper airway) for more than a single substantial cross sectional region, or partial cross section and thus allows a more realistic analysis of the volume to be made without the need to move the catheter or endoscope within the volume.

The light pattern generator may be described as a structured light source. The pattern for example has at least one narrow peak in intensity along the catheter direction. This peak may be continuous around the full radial direction (i.e. an annular ring) or it may be discontinuous around the radial direction (i.e. a set of spots). The intensity preferably drops as close to zero as possible each side of the narrow peak.

The light pattern generator may further comprise: a lens configured to generate a collimated or partially collimated beam substantially aligned along the sensor length; and a reflective element configured to reflect the light beam to generate the at least one light pattern at an oblique and/or right angle to the sensor length.

In such embodiments, the length of the sensor can be shortened as the reflective element may convert an axial light pattern into a radial light pattern. Furthermore such a sensor is specifically able to operate within and/or along a catheter as the generation of the at least one light pattern at a radial projection prevents a proximal or distal part of the catheter from shadowing the sensors operation.

The first light redirection element may comprise a first reflective element and the second light redirection element may comprise a second reflective element.

The first reflective element may comprise a reflective cone comprising a single reflective surface angle configured to generate the at least one light pattern in the form of a ring at an oblique and/or right angle to the sensor length.

In such embodiments the reflective cone may convert an axial light pattern into a radial light pattern where the beam is reflected to form a ring pattern which can be reflected onto the inner surface of the elongated volume.

The first reflective element may comprise a reflective cone comprising a stepped reflective profile having at least two different reflective surface angles and configured to generate at least two light patterns in the form of at least two rings at an oblique and/or right angle to the sensor length.

In such embodiments the stepped reflective profile by generating at least two light patterns in the form of at least two rings may permit the easier detection and determination of the cross sectional profile of the elongated volume.

The first reflective element may comprise a reflective cone comprising a varying reflective surface angle and configured to generate a distributed light pattern at an oblique and/or right angle to the sensor length.

Similarly in some embodiments by generating a distributed light pattern the detection and determination of the cross sectional profile of the volume may be assisted.

The first reflective element may comprise a diffractive optical element within the optical pathway of the light beam either before or after a reflective cone configured to generate the at least one light pattern at an oblique and/or right angle to the sensor length based on the diffractive optical element.

In such embodiments the diffractive optical element enables the generation of a more sophisticated pattern, such as multiple rings, which may allow a more accurate cross sectional determination to occur.

The first reflective element may comprise a volume hologram within the optical pathway of the light beam configured to generate the at least one light pattern at an oblique and/or right angle to the sensor length based on the diffractive optical element.

The imaging device substantially aligned along a length of the optical sensor may comprise a camera located on the side of and external of the optical sensor and directed along the direction of the light beam.

In such arrangements the camera may capture images of the light pattern projected on the volume (airway) wall in a forwards (or axial) field of view.

The imaging device substantially aligned along a length of the optical sensor may comprise a camera located within the optical sensor and directed along and substantially opposite the direction of the light beam.

In such arrangements the camera may capture images of the light pattern projected on the volume (airway) wall in a forwards (or axial) field of view, but allows the diameter of the catheter sensor to be reduced as there is no ‘stacking’ of the components in a radial direction.

The imaging device substantially aligned along a length of the optical sensor may comprise a camera and the second reflective element located within the optical sensor and directed along and substantially opposite the direction of the light beam, the reflective element configured to reflect at least part of the field of view of the camera from an axial field of view direction to a radial field of view direction.

The second reflective element may comprise a reflective cone comprising a single reflective surface angle configured to reflect at least part of the field of view of the camera (1013) from an axial field of view direction to a radial field of view direction.

In such arrangements the camera may capture images of the light pattern projected on the volume (airway) wall in a sideways (or radial) field of view which assists in reducing the length of the sensor.

The second reflective element may comprise a reflective cone comprising a stepped reflective profile having at least two different reflective surface angles and configured to reflect a first part of the field of view of the camera from an axial field of view direction to a first range radial field of view directions, and a second range of the field of view of the camera from an axial field of view direction to a second range radial field of view directions non continuous with the first range radial field of view directions.

In such arrangements the camera may capture images of the light pattern projected on the volume (airway) wall in a sideways (or radial) field of view which assists in reducing the length of the sensor but allows an overlapping field of view of the camera to assist in determining any erroneous images.

The second reflective element may comprise a reflective cone comprising a varying reflective surface angle and configured to generate a sensor field of view range greater than the field of view of the camera

In such arrangements the camera may capture images of the light pattern projected on the volume (airway) wall with a greater degree of coverage that would be provided by the original camera.

The second reflective element may comprise a reflective cone comprising a varying reflective surface angle and configured to generate a sensor field of view range less than the field of view of the camera.

In such arrangements the camera may capture images of the light pattern projected on the inner wall of the volume (airway) with a smaller degree of coverage that would be provided by the original camera but with a greater density of imaging of the area that is covered.

The optical sensor may further comprise a transparent capillary configured to support the light pattern generator and the imaging device and further permit the transmission of the at least one light pattern from optical sensor to the inner surface of the volume.

In such embodiments the transparent capillary may support and protect the pattern generator and imaging device from mechanical damage, and enable easy cleaning of the sensor.

The first reflective element may comprise a solid body having an internal cavity which defines a cone, the internal cavity having a lower refractive index than the material of the solid body. This enables a simple manufacturing process, and it makes use of total internal reflection from the higher refractive index material of the solid body to the lower refractive index of the cavity (e.g. air).

The internal cavity may define the first and second reflective elements facing in opposite directions. The two reflective elements are thus formed from a single block thereby reducing cost.

Whether formed as a block or not, the first and second reflective elements are may be back-to back with their reflecting surfaces facing outwardly. This enables the diameter to be reduced as the reflectors and the imaging device and illumination source are located at different axial positions.

In an alternative design, the first reflective element is offset from a central axis of the optical sensor and the first light beam is offset from the central axis, and the second reflective element is centered on the central axis, wherein the first and second reflective elements face in the same direction. This provides a more compact arrangement axially, while maintaining separate reflectors (e.g. cones) for the light pattern generation and for the collection of reflected light.

The optical sensor may further comprise at least one transparent rod, wherein the at least one transparent rod may comprise at least one of: a lens hole or hollow configured to receive a light guide and configured to operate as a lens configured to generate a light beam substantially aligned along the sensor length; a light pattern generator hole or hollow configured to reflect the light beam to generate the at least one light pattern; a field of view hole or hollow configured to reflect at least part of the field of view of the imaging device from an axial field of view direction to a radial field of view; an imaging device hole or hollow configured to receive the imaging device.

Thus in such embodiments the transparent rod may support and protect the pattern generator and imaging device from mechanical damage, and enable easy cleaning of the sensor and furthermore provide a structure which reduces the number of optical interfaces and therefore reduces the number of parasitic reflections captured by the imaging device.

The at least one transparent rod may comprise: a first transparent rod comprising the lens hole or hollow and light pattern generator hole or hollow; a second transparent rod comprising the field of view hole or hollow and the imaging device hole or hollow, wherein the first transparent rod and the second transparent rod are fixed together. The first transparent rod and the second transparent rod may be fixed together by glue.

This arrangement in some embodiments allows the easy construction of the light pattern generator hole or hollow and the field of view hole or hollow, by forming them in the ends of the two transparent rods before gluing the rods together.

The optical sensor may be rigid member such that the optical distance between the light pattern generator and the imaging device is a defined length.

In such a manner a fixed optical distance between the light pattern generator and the imaging device enables the determination of the cross sectional distances to be performed using simple geometric determination.

The optical sensor may further comprise at least one conduit, the conduit may be further configured to locate within the sensor at least one light guide for at least one further optical sensor.

In such a manner the sensor may be arranged with further sensors and provide a light source to sensors located at the distal end of a catheter arrangement.

The optical sensor may further comprise at least one light source, the at least one light source may be optically coupled to the lens.

The at least one light source may comprise at least one of: at least one light emitting diode; at least one laser diode; at least one vertical-external-cavity surface-emitting-laser.

The optical sensor may further comprise at least one conduit, the conduit may be further configured to locate within the sensor at least one imaging device output from at least one further catheter sensor.

In such a manner the sensor may be arranged with further sensors and provide a data pathway from sensors located at the distal end of a catheter arrangement.

A catheter may comprise at least two optical sensors as described herein, the at least two optical sensors being distributed spaced along the catheter, and wherein the at least two optical sensors are configured to observe different substantial cross sections of a volume within which the catheter is located.

According to a second aspect there is provided an imaging method for obtaining images from an optical sensor provided along a catheter, wherein the sensor is configured to observe a cross section of an elongated volume within which the catheter (103) is located, the optical sensor having a sensor length along a direction parallel to the catheter direction, the method comprising:

projecting at least one light pattern at a radial projection angle with respect to the optical sensor length onto the inner surface of the elongated volume into which the optical sensor is inserted, the projecting comprising providing a first light beam having a component of its direction along the sensor length and using a first light redirection element to redirect the light beam to generate the at least one light pattern at an oblique and/or right angle to the optical sensor length;

redirecting a reflected version of the light pattern from the inner surface of the elongated volume using a second light redirection element to provide a second light beam; and

receiving the second light beam using an imaging device having a field of view with a central axis substantially parallel to the length of the optical sensor.

Projecting at least one light pattern at a radial projection angle to the catheter on the inner surface of the elongated volume, may further comprise: generating a light beam substantially aligned along the sensor length; and reflecting the light beam to generate the at least one light pattern at an oblique and/or right angle to the sensor length.

The light source used to generate the illumination pattern may comprise at least one of: a light emitting diode; a laser diode; a vertical-external-cavity surface-emitting-laser. A conduit may be provided within or on the optical sensor, the conduit locating at least one of: at least one light guide for at least one further optical sensor; and at least one imaging device output from at least one further catheter sensor.

A catheter may comprise at least two optical sensors as described herein, the at least two optical sensors being distributed spaced along the catheter, and wherein the at least two optical sensors are configured to observe different substantial cross sections of a volume within which the catheter is located.

The volume may be an upper airway volume.

The substantial cross sections of diagnostic interest within the volume may be the velum, oropharynx, base of tongue and epiglottis.

### BRIEF DESCRIPTION OF THE DRAWINGS

Examples of the invention will now be described in detail with reference to the accompanying drawings, in which:

FIG. 1 shows a typical upper airway and example cross sections;

FIG. 2 shows a first example of an upper airway catheter according to some embodiments;

FIG. 3 shows a detail of the first example of an upper airway catheter according to some embodiments;

FIG. 4 shows the first example upper airway catheter sensor configuration as shown in FIGS. 2 and 3 according to some embodiments;

FIG. 5 shows a upper airway monitoring system incorporating an upper airway catheter as shown in FIGS. 2 to 4 and a data processing unit according to some embodiments;

FIG. 6 shows an example of a collapse event as monitored by the upper airway monitoring system shown in FIG. 5;

FIG. 7 shows an example ultrasonic sensor as implemented on the upper airway catheter as shown in FIGS. 2 to 4 according to some embodiments;

FIG. 8 shows a first example optical sensor as implemented on the upper airway catheter as shown in FIGS. 2 to 4 according to some embodiments;

FIG. 9 shows a second example optical sensor as implemented on the upper airway catheter as shown in FIGS. 2 to 4 according to some embodiments;

FIG. 10 shows an alternative way to convert an axial illumination pattern to a radial illumination pattern;

FIG. 11 shows an example cross sectional dimension determination based on the second example optical sensor shown in FIG. 9;

FIG. 12 shows a third example optical sensor as implemented on the upper airway catheter as shown in FIGS. 2 to 4 according to some embodiments;

FIG. 13 shows two examples of field of view reflection suitable for implementing in the third example optical sensor as shown in FIG. 11;

FIG. 14 shows a fourth example optical sensor as implemented on the upper airway catheter as shown in FIGS. 2 to 4 according to some embodiments;

FIG. 15 shows examples of light patterns which are suitable for implemented in example optical sensors as shown in FIGS. 8 to 14 according to some embodiments;

FIG. 16 shows an example of a curved profile reflective element suitable for implementation in example optical sensors as shown in FIGS. 8 to 14 according to some embodiments;

FIG. 17 shows an example of a stepped profile reflective element suitable for implementation in example optical sensors as shown in FIGS. 8 to 14 according to some embodiments;

FIG. 18 shows a fifth example of an optical sensor as implemented on the upper airway catheter according to some embodiments of the invention;

FIG. 19 shows a second example of an upper airway catheter sensor configuration according to some embodiments; and

FIG. 20 shows a third example of an upper airway catheter sensor configuration according to some embodiments.

### DETAILED DESCRIPTION OF THE EMBODIMENTS

The concept as embodied in the description herein is an optical sensor which may be applied to a flexible or semi-flexible catheter which can be placed into the upper airway of a patient to measure changes of airway geometry during natural sleep. This catheter may comprise at least two sensors which are distributed spaced along the catheter, wherein the sensors are configured to measure or observe the different substantial cross sections of a volume within which the catheter is inserted, which in some embodiments is the local cross section of the airway. In some embodiments the locations of these sensors may be aligned with key anatomical locations or locations of diagnostic interest (for example the sensors are aligned with the velum (V), oropharynx (O), base of tongue (T), and epiglottis (E) when the catheter is inserted). In some embodiments the number and spacing of the sensors is such that it is possible to directly measure or determine an airway cross-section measurement at each of these key anatomical locations. In some embodiments the number and spacing of the sensors is such that it is possible to interpolate the airway cross section at these key anatomical locations where the key location occurs between sensors. From the change of cross section a data processing unit may be configured to calculate key physiological parameters like the percentage of narrowing and/or configuration of narrowing (for example whether the narrowing is an anterior-posterior narrowing, a lateral narrowing or a circular or radial narrowing). The data processing unit may in some embodiments comprise a user interface (UI) configured to present the data of the full night measurement to the ENT specialist in a useable format. For example in some embodiments the UI may be configured to generate an accepted upper airway classification method like VOTE, or be configured to replay the recorded key events. In some embodiments the system may be integrated with other sensors (for example body position, oxygen saturation, sleep stage) or in some embodiments be used during a full polysomnography (PSG) study. In such a manner the system can provide correlations between airway geometry and sleep parameters, for example the dependence of obstructions patterns on sleep stage/position or which collapse patterns cause the strongest oxygen desaturation events. In some embodiments the individual sensors are optical (patterned light) sensors or ultrasound sensors to measure the airway cross section, as will be described below. A substantial cross section is one in which the majority of the cross section of the inserted volume is observed. For example a substantial cross section in some embodiments is one where more than 180 degrees (or pi radians) of cross section is being observed. Thus for example a full cross section of 360 degrees (or 2 pi radians) or nearly full cross section of between 270 to 360 degrees (or 3/2 pi to 2 pi radians) observation is the goal for embodiments as described herein. It would be understood that a substantial cross section is understood as not being limited to only the meaning of providing a substantial coverage observation of the cross section but also including the meaning of providing a substantial range of observation of the cross section. The cross section may extend along the volume, so that it is not only a thin line.

In other words the observation can in some embodiments be one of observing of a substantially continuous ring pattern (of light) on the volume interior wall. In such embodiments the substantial cross section coverage and range is provided directly by the observation. However a substantial cross section range can be achieved in some embodiments by a broken ring pattern (of light) being observed by a substantially continuous sensor, or a substantially continuous ring pattern (of light) observed by a non-continuous sensor or sensor array, or a broken ring pattern (of light) observed be a non-continuous sensor or sensor array but providing a substantial cross section range. In such embodiments the substantial coverage can be generated or determined by interpolation. For example where in some embodiments the gaps in observed coverage are small and/or regularly distributed these values can be ‘filled in’ or interpolated easily.

It would furthermore be understood that the optical and/or other sensors can be configured to observe or visualize more than only this projected ring. For example the sensors can be configured to observe the substantial cross-section to provide more information about the surface of the inner airway wall. For example whereas an optical sensor observing a single ring may provide a one-dimensional ‘view’, in other words only the ring image, the sensor can be configured in some embodiments to provide a two-dimensional or area ‘view’ with a substantial cross-section. For example this could be provided by an array of neighboring rings and interpolating the space between them or by the optical sensor or another sensor (such as an ultrasound sensor) observing or imaging a two-dimensional area of substantial cross section range.

Furthermore in the embodiments as described herein the sensor and in particular the optical sensor as described herein is configured to be located within and along the catheter. As such the optical sensor is configured to generate or project (by a light pattern generator) at least one light pattern at a radial projection angle to the (local) catheter length direction onto an inner surface of an elongated volume into which the optical sensor is inserted. It would be understood that a radial projection angle is possibly a right angle from the local catheter length direction but may also be any suitable oblique angle with respect to the local catheter length direction and preferably one within a right angle sector centered at the normal of the catheter length direction and therefore the sensor length direction.

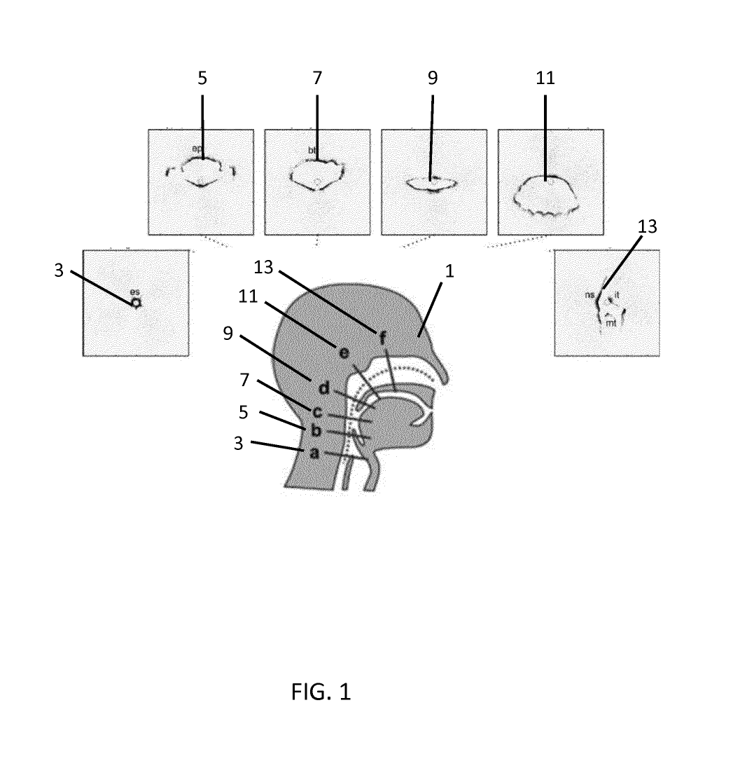

With respect to FIG. 1 an example patient upper airway is shown with typical cross sections at designated locations within the upper airway. The patient 1 is shown with cross sections locations such as the Esophagus 3, Hypopharynx (or Laryngophyarynx or epiglottis) 5, Oropharynx (or base of tongue) 7, Velopharynx 9, Proximal nasopharynx 11 and Nasal cavity 13. The anterior-posterior direction in the example cross sections is the vertical, and the lateral direction is the horizontal.

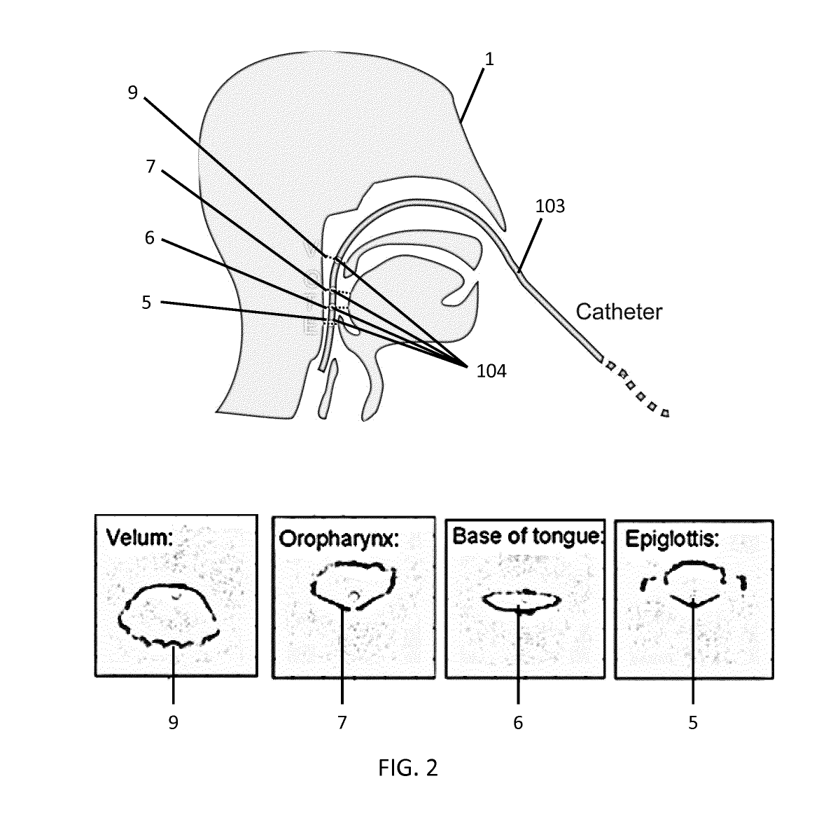

Furthermore with respect to FIG. 2 a first example of an upper airway catheter 103 is shown in location according to some embodiments (in other words the catheter 103 following insertion into the patient). The catheter 103 may in some embodiments be inserted through the patient's 1 nose until the caudal side is lodged in the esophagus. The insertion may be performed by a trained nurse or an ENT specialist in the evening or before a nap during the day.

FIG. 2 further shows examples of cross sections as measured with a catheter 103 at the defined locations of the velum (or the soft palate) 9, the oropharynx 7, the base of the tongue 6 and the epiglottis 5. The anterior-posterior direction in the example cross sections is the vertical; and the lateral direction is the horizontal.

The catheter 103 may comprise a multitude of sensors 104 configured to measure the cross section at the upper airway at (or near) defined structures or locations within the upper airway. In some embodiments these structures or locations to be observed and monitored may comprise the velum (or the soft palate) 5, the oropharynx 7, the base of the tongue 9 and the epiglottis 11.

The catheter 103 may be flexible in that the whole length of the catheter 103 can move or bend. However it would be understood that in some embodiments the catheter 103 is configured to be semi-flexible in that parts or portions of the catheter 103 are flexible and other parts or portions of the catheter 103 are rigid or non-flexible, or be semi-flexible in that some dimensions of portions of the catheter 103 are flexible and some dimensions are fixed or rigid.

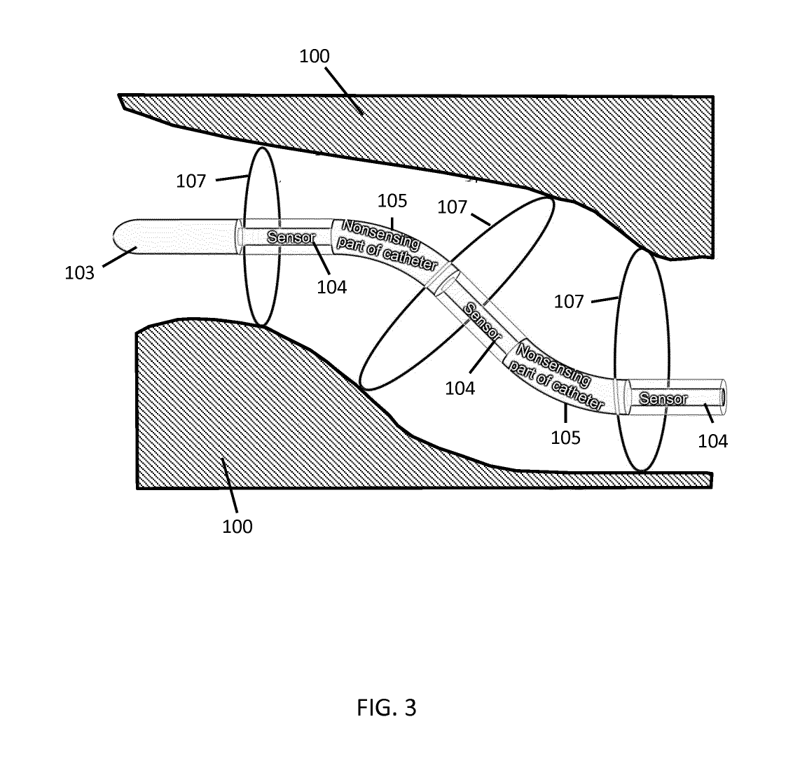

For example FIG. 3 shows an example catheter 103 comprising flexible non-sensing parts 105 located between rigid sensor 104 parts. The rigid sensor 104 parts of the catheter may then be configured to determine cross-sectional profiles 107 as defined by the upper airway walls 100.

In some embodiments the catheter 103 may be steerable in that the flexible parts 105 or flexible catheter 103 can be actively directed during insertion. In some embodiments the catheter 103 may be passively directed during insertion in that it flexes or bends in response to coming into contact with the upper airway walls 100.

To lessen patient discomfort the diameter of the catheter 103 may be less than 5 mm and may be less than or equal to 3 mm in diameter.

In some embodiments in order to better monitor these structures for different patients with different airway lengths, the location of the sensors 104 may be adaptable. In such embodiments the ENT specialist may configure the catheter 103 for each patient based on prior measurements. In other words in some embodiments the spacing of the catheter 103 sensors 104 can be adjusted relative to each other. For example in some embodiments the catheter 103 may comprise telescopic or adjustable length parts or portions between the sensors to adjust the relative locations of the sensors 104. In some embodiments the sensors 104 themselves may be movable on the catheter 103 body. In some embodiments there may be catheters 103 of various lengths (for example short, medium and long) that can be chosen according to the patient upper airway length.

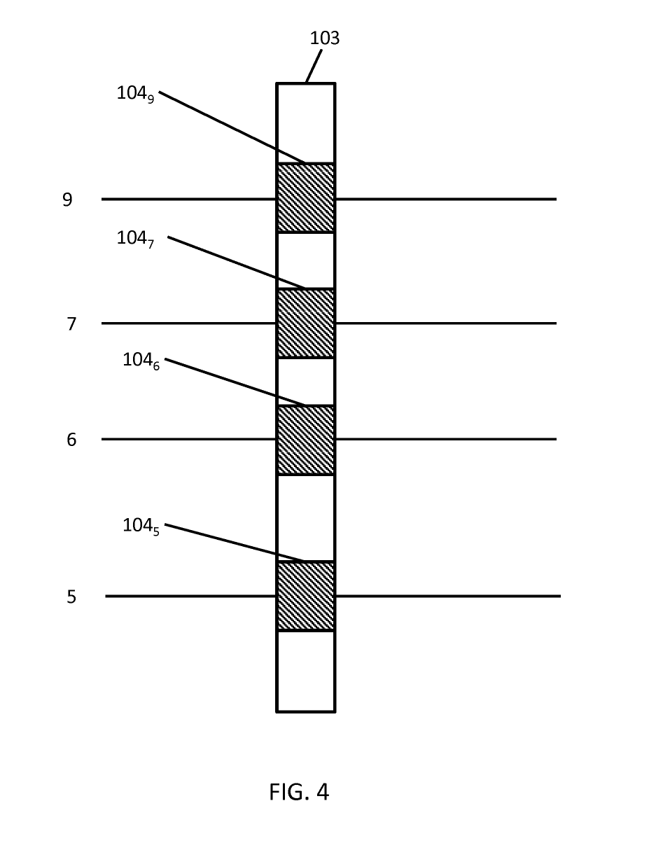

With respect to FIG. 4 a configuration of the first example catheter 103 according to some embodiments is further shown. The catheter 103 as described here comprises a first or velum sensor 104₉ configured to be located within the velum 9 region of the patient's upper airway, a second or oropharynx sensor 104₇ configured to be located within the oropharynx 7 region of the patient's upper airway, a third or base of tongue sensor 104₆ configured to be located within the base of tongue 6 region of the patient's upper airway, and a fourth or epiglottis sensor 104₅ configured to be located within the epiglottis 5 region of the patient's upper airway.

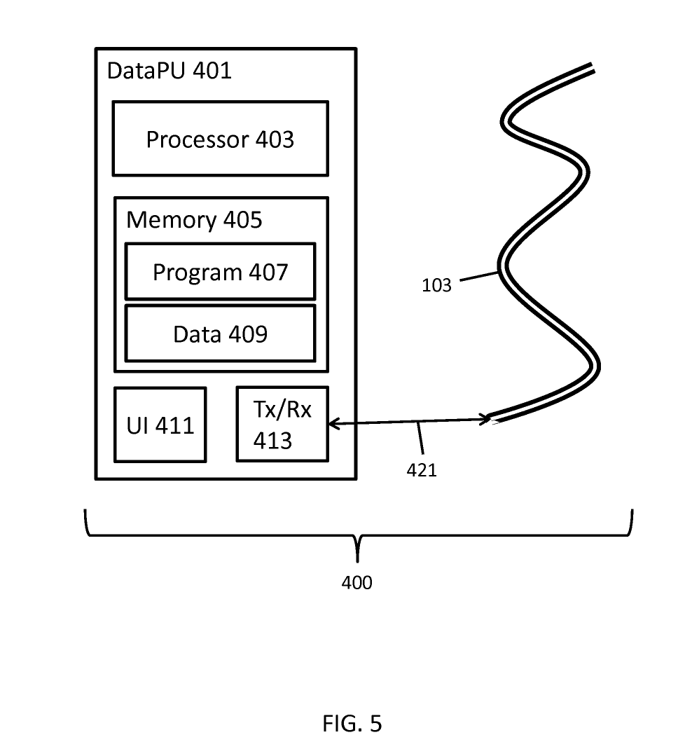

With respect to FIG. 5 an example of a catheter or monitoring system 400 suitable for operating the catheter 103 is shown. The catheter system 400 may in some embodiments comprise the catheter 103, such as shown in FIGS. 2 to 4, which may in some embodiments be connected to a data processing unit (DataPU) 401, and in some embodiments an interface or transceiver (Tx/Rx) 413 within the data processing unit 401 via an interface coupling 421. The data processing unit 401 may comprise a processor 403 configured to receive the sensor 104 data and determine or generate suitable cross sectional information. The data processing unit 401 may furthermore comprise at least one memory 405, which in some embodiments may be sub-divided into program memory 407 configured to store instructions for operating or execution by the processor 403, for example programs or instructions for processing sensor data to determine the cross-sectional information or results. The at least one memory 405 may furthermore in some embodiments comprise data memory 409 configured to store data such as for example unprocessed sensor data. Furthermore in some embodiments the data memory 409 may in some embodiments be configure to store processed data such as the cross sectional information determined by the processor 403 during the duration of the patients sleep.

The data processing unit 401 may in some embodiments comprise a user interface (UI) 411. The user interface 411 may be any suitable user interface, for example a touch screen display configured to enable the display of data to the user of the system and also data input from the user. In some embodiments the user interface 411 may comprise separate data display and data input means. Thus for example the data processing unit 401 may comprise a keyboard/keypad for entering data inputs and a display screen for displaying data to the ENT.

In some embodiments the user interface 411 may be configured to present the data stored within the data processing unit 401 of the full night's measurement to the ENT specialist in a useable format. For example in some embodiments the UI 411 may be configured to generate an accepted upper airway classification method like VOTE, or be configured to replay any determined (recorded) key events.

In some embodiments the data processing unit 401 may be further configured to receive data from other sensors. For example in some embodiments the data processing unit may be configured to receive data from sensors such as body position, oxygen saturation, sleep stage. This other sensor data may further be time coded or synchronized with the catheter 103 based sensor 104 data such that the data processing unit 401 may be configured in some embodiments to determine and/or display any correlations between airway geometry and sleep parameters, for example the dependence of obstructions patterns on sleep stage/position or which collapse patterns cause the strongest oxygen desaturation events.

In some embodiments the catheter system 400 comprising the data processing unit 401 and the catheter may be employed during a full polysomnography (PSG) study.

In some embodiments the data processing unit 401 may be configured to determine from the cross sectional information from the sensors 104 a number of clinical relevant parameters. For example in some embodiments the data processing unit 401 may be configured to determine the cross sectional area, and from which the data processing unit 401 can further determine the percentage of narrowing. Furthermore in some embodiments the data processing unit 401 may be configured to determine the configuration of an upper airway collapse event. For example whether the upper airway collapse event is a predominantly anterior-posterior (AP), lateral, or circular collapse.

It would be understood that in order that any measurements to reflect events which may occur during natural sleep it would be important that the catheter is not moved from the outside as this would impair the patient's sleep. However in some embodiments the data processing unit 401 may be configured to operate in a ‘scan’ or ‘insertion’ mode during insertion where a 3D image of the airway is acquired as the catheter is inserted. The 3D image in such embodiments may be used to provide precise information on where in the airway the sensors are located during the subsequent full night measurement. Also, as the catheter position may change slightly over time (for example due to the patient's movement), in some embodiments the data processing unit 401 may be configured to detect such dislocations of the sensors and in some embodiments compensate for these movements.

In some embodiments to determine a full airway image it may be necessary to initially insert the catheter 103 deeper than necessary for the full-night measurement and then retract it for the measurement. Furthermore in some embodiments the catheter 103 determines or generates an insertion depth measurement during the insertion and measurement operations.

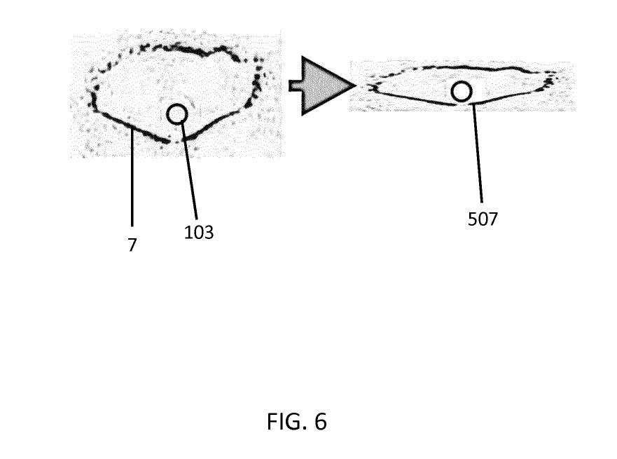

An example of an anterior-posterior collapse event as determined and displayed by the data processing unit 401 is shown in FIG. 6, wherein a pre-collapse oropharynx 7 image is shown with the catheter 103 in situ on the left hand side and an anterior-posterior collapsed oropharynx 507 image shown with the catheter in situ on the right hand side

The data processing unit 401 may in some embodiments prepare the data in a form that facilitates the reception by the ENT. This can for example be a summary form comprising information showing how many collapse events occurred, the type or configuration of the collapse events, when the collapse events occurred and whether there is any correlation between the collapse event and any other sensor data. Thus in some embodiments the data processing unit 401 may access data from additional sensor(s) to provide ‘richer’ event data, such as determining the type of collapse based on the sleep stage or a typical oxygen desaturation measurement for a type of collapse.

In some embodiments the data processing unit 401 may generate or determine a ‘3D’ model representing the change of the airway shape during any determined collapse events. The data processing unit 401 may furthermore enable the user of the system, for example the ENT, the ability to replay certain events.

The data processing unit 401 can thus, in some embodiments, be configured to construct a model of the volume. The model of the volume may be generated based on the data provided by the sensors located on the catheter 103. The data processing unit 401 may furthermore be configured (based on the model of the volume or on the data provided by the sensors located on the catheter 103) to generate clinical information suitable for analysis by the ENT. The clinical information in some embodiments comprises determining at least one volume contraction or collapse. Furthermore the data processing unit 401 can in some embodiment based on determining the at least one volume contraction or collapse be configured to generate further clinical information such as determining at least one of: the location of the at least one contraction or collapse; the degree (severity) of the at least one volume contraction or collapse; and the configuration of the at least one volume contraction or collapse.

The configuration of the at least one volume contraction or collapse is the known term which described the dominant direction of the contraction or collapse. For example a dominant direction of the contraction or collapse may as described herein be an anterior-posterior (AP) contraction or collapse, a lateral contraction or collapse, or a circular contraction or collapse.

It would be understood that in some embodiments the data processing unit 401 can be configured to generate such clinical information over a period of time. For example the data processing unit 401 can be configured to monitor or determine the clinical information over a night or ‘full’ night.

Furthermore it would be understood that in some embodiments the data processing unit can be configured to sort the clinical information over a sub-period of time. For example a suitable sub-period of time may be a sleep state period and/or a sleep position period.

As described herein the data processing unit 401 furthermore can be configured to store the clinical information, the at least one volume collapse (and the associated characteristics or descriptors of the at least one volume collapse or contraction such as the location, the degree (severity), and the configuration of the at least one volume collapse or contraction).

Similarly the data processing unit may be configured to replay the stored at clinical information.

The user interface as described herein can for example display the determined/stored/replayed clinical information. For example the user interface can be configured to display the clinical information in the form of the at least one volume collapse (and the associated characteristics or descriptors of the at least one volume collapse or contraction such as the location, the degree (severity), and the configuration of the at least one volume collapse or contraction) can be displayed.

The sensor 104 for such an airway catheter 103 faces a number of constraints that require significant design skill in order to achieve a successful result.

As discussed herein the catheter 103 and therefore the sensor 104 diameter has to be <=5 mm and preferably <=3 mm to ensure patient acceptance. Furthermore where in some embodiments the sensor is rigid and not flexible, the sensor length has to be limited to ensure passage of the sensor through the nose. The length of a rigid sensor should therefore in some embodiments not exceed 2 cm and preferably the length of a rigid sensor should be <=1 cm.

As shown with respect to the catheter 103 in FIGS. 2 to 5 the catheter has to be configured in such a way that multiple sensors are integrated into (the middle of) a flexible catheter. This for example prevents a typical optical fiber endoscope design being implemented for all of the sensors 104 as the view in the forward direction would be blocked by the catheter in the sensors located along the length of the catheter 103.

In some embodiments any sensor 104 connections to the outside are configured to be small enough not to conflict with or interfere with other sensors 104.

In some embodiments the sensor 104 is configured to be encapsulated, and therefore easily cleanable. Similarly the sensor 104 is configured in some embodiments such that a thin coat of saliva or slime should not prevent the sensor from functioning or from producing inaccurate data. The sensor 104 in some embodiments is configured to operate at a rate fast enough to detect a collapse. Similarly in some embodiments the sensor 104 is configured to operate at a rate fast enough to detect (and enable the data processing unit 401 in some embodiments to filter out) other movements of the airway, such as for example breathing motion.

The sensor 104 in some embodiments is configured to operate without the need for mechanical scanning or movement. This is because any movement of (or inside) the catheter 103 can keep the patient from falling asleep or wake the patient up and because mechanical arrangements tend to make a sensor fragile.

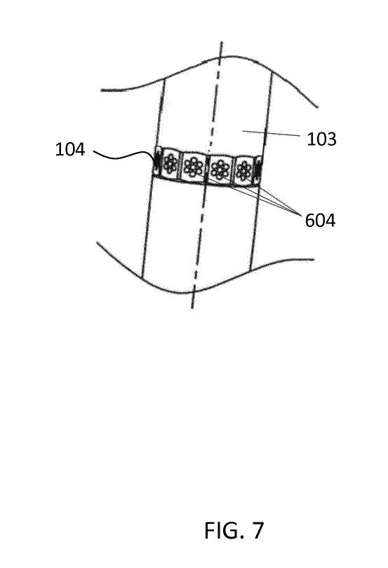

With respect to FIG. 7, a first example sensor is shown. In some embodiments the sensor configured to measure the cross sectional dimension of the airway is at least one or an array of ultrasound transducers 604 arranged around the catheter 103. In order to keep the diameter of the catheter as small as possible the ultrasound transducer 604 may be, in some embodiments, ‘small’ ultrasound transducers, for example CMUTs (capacitive micro-machined ultrasonic transducers). CMUTs 604 may in some embodiments be manufactured on a flexible substrate and thus in some embodiments the sensor 104 comprises a ring of ultrasound transducers 604 around the catheter 103 configured to determine the cross section of the upper airway at a number of directions. It would be understood that the ultrasound transducer(s) 604 would be impedance matched to the medium (typically air in the upper airways) to enable optimal out coupling of the acoustical waves.

In some embodiments the sensor 104 may be implemented by an optical sensor that fulfills the requirements above.

The optical sensor as described herein may be configured to comprise an optical element that generates a light pattern (for example a light ring), one or more reflective elements (for example reflective cones) to direct the light pattern and/or the field of view (FOV) of the imaging device (for example a camera) to the catheter sides (it would be understood that in some embodiments the reflective element may be integrated into the light pattern generating element), an imaging device (for example a miniature camera with a large field of view).

In some embodiments in order to be able to determine a cross sectional measurement the optical element, the reflective element(s) and the imaging device have a fixed geometric relationship.

Furthermore in some embodiments the optical element configured to generate the light pattern is connected to a laser diode via an optical fiber. In other embodiments a laser diode may be incorporated inside the sensor.

In some embodiments as described herein the optical fiber, the optical element, the reflective element(s) and the imaging device are all integrated into a plastic or glass element that is (partly) transparent to allow the light pattern to illuminate the upper airway and the imaging device (camera) to see the light pattern on the airway walls and which protects the sensor.

Furthermore in some embodiments the output of the imaging device is processed in order to obtain the airway cross section information.

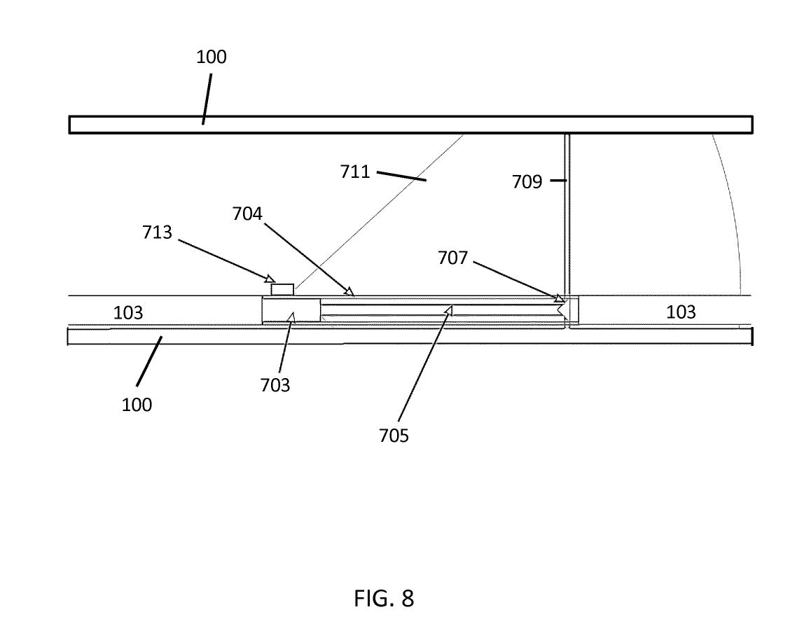

With respect to FIG. 8, a cross sectional view of an example optical sensor is shown. In some embodiments the example optical sensor is configured to measure the cross sectional dimension of the airway by generating a light pattern (e.g. a ring) within the airway, which is captured from a different position by an imaging device (for example by a small camera). In such a manner in some embodiments the image of the projection of the light pattern on the airway wall can enable the reconstruction of the airway shape by the use of the fixed geometry between the generated light pattern and imaging device.

The optical sensor as shown in FIG. 8 is located within a transparent capillary 704 within the catheter 103. Furthermore the optical sensor comprises an optical fiber which is configured to transmit a light beam to a gradient index (GRIN) lens 703 located at the fiber end. The GRIN lens 703 (or any other suitable lens configuration) is configured to produce a light beam (laser beam) 705 which is projected within the transparent capillary 704 onto a surface of a reflective cone 707 also within the transparent capillary. The reflective cone 707 is configured to reflect the light such that it passes through the transparent capillary 704 and generates a ring pattern 709 which projected onto the airway wall 100. An imaging means or device such as a small camera 713, which is shown in FIG. 8 located outside of the capillary 704, is aligned substantially in the same direction as the light beam 705. It is configured with a field of view 711 which is directed generally along the same direction as the light beam, i.e. parallel to the catheter axis and accordingly parallel to a general elongate axis of the optical sensor. Thus, the camera has a field of view which is directed generally along this direction, namely it has a central axis substantially parallel to the catheter elongate direction. By virtue of the angular width of the field of view of the camera, it is enabled to capture an image comprising the projection of the ring pattern 709 on the airway wall 100. The image generated by the imaging device or camera 713 can be furthermore configured to pass the image data to the data processing unit 401.

The data processing unit 401, having received the image data, and with the determined geometric relationship between the optical element (the lens 703), the reflective element (the reflective cone 707) and the imaging device (the camera 713 and the camera field of view 711) can be configured to analyze the captured image data and the ring pattern to determine (or reconstruct or generate) the airway cross section dimensions.

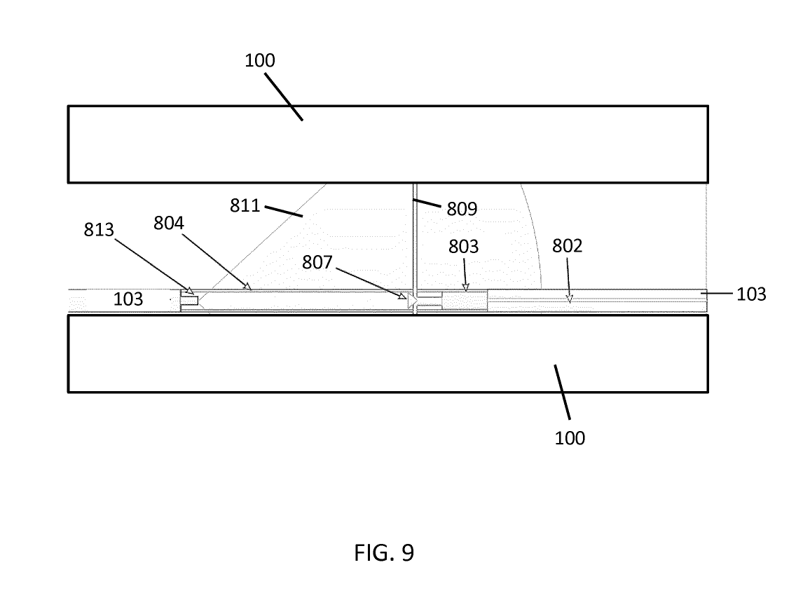

With respect to FIG. 9, a cross sectional view of a second example optical sensor is shown. The second example optical sensor differs from the first example optical sensor in that the imaging device or camera 813 is located within the transparent capillary or glass tube 804 which enables the diameter of the sensor to be reduced and produces an image without ‘blind areas’ on the other side of the catheter 103 to the location of the imaging device (in other words does not require the catheter 103 to be orientated in a defined or specific direction to prevent shadowing of the imaging device by the catheter).

The optical sensor as shown in FIG. 9 is located within a transparent capillary or glass tube 804 within the catheter 103. Furthermore the optical sensor comprises an optical fiber 802 which is configured to transmit a light beam to a gradient index (GRIN) lens 803 located at the fiber end. The GRIN lens 803 (or any other suitable lens configuration) is configured to produce a light beam (laser beam) which is projected within (and generally in the direction along) the glass tube 804 onto a surface of a reflective cone 807 also within the glass tube 804. The reflective cone 807 is configured to reflect the light such that it passes through the glass tube 804 wall and generates a ring pattern 809 which projected onto the airway wall 100. An imaging means or device such as a small camera 813 which is shown in FIG. 9 located within the glass tube 804 is aligned substantially in the opposite direction as the path of the light beam. It is again configured with a field of view 811 and enabled to capture an image comprising the projection of the ring pattern 809 on the airway wall 100. The image generated by the imaging device or camera 813 can be furthermore configured to pass the image data to the data processing unit 401.

The first and second examples of optical sensors as shown herein with respect to FIGS. 8 and 9 can be considered to be ‘forward looking sensors’ in that the imaging devices are generally in alignment with the catheter 103 and therefore in alignment with the transparent capillary or glass tube arrangement. They both use a reflecting cone to redirect the pattern generation light from an axial direction to a radial direction.

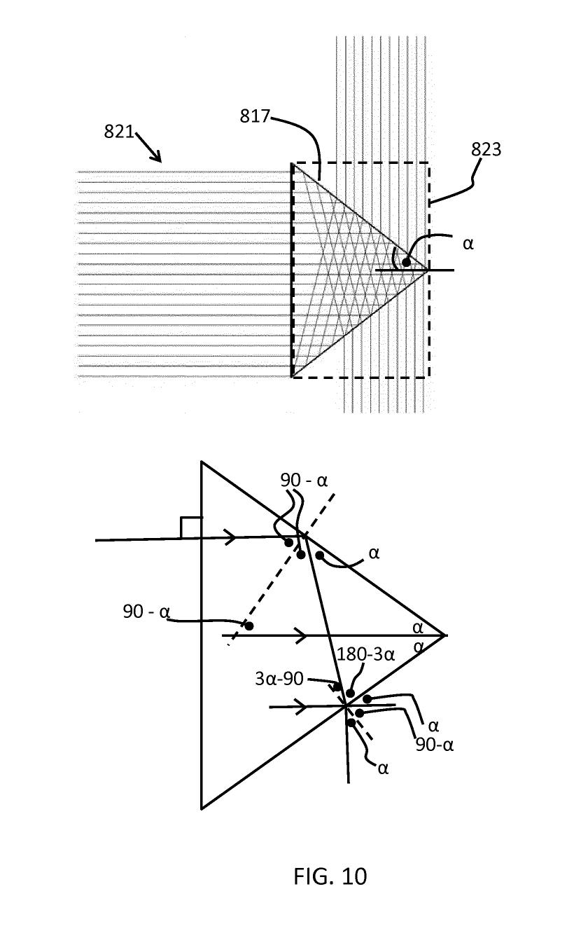

An alternative approach shown in FIG. 10 makes use of total internal reflection. Thus, the term “reflective element” used in this application should be understood as not being limited to specular reflection but includes total internal reflection.

An optical cone 817 is again provided, but it comprises a transparent material having a higher refractive index than the surroundings. The incident light 821 directed along the catheter axis direction is provided to the base of the cone (perpendicular to the catheter axis direction). The light experiences no refraction as the air to cone interface is perpendicular.

The light then experiences total internal reflection at the internal cone to air boundary defined by the tapered conical face. This is because the cone angle and refractive index of the cone material are chosen to give rise to total internal reflection at the conical internal face. After reflection, the light passes to a radially opposite part of the internal cone face, with an incident angle closer to the normal. The light then exits the cone, in a direction (after a refraction at the interface which bends the light away from the normal) which is at 90 degrees to the original incident direction. This 90 degree angle is not essential, and the exit direction may instead not be perfectly radial.

The incident light 821 is for example again received from a graded refractive index lens at the cleaved facet end of an optical fiber. This lens may have a width of around 0.25 mm.

The cone 817 is formed of a body of refractive index n and has a top cone half angle α which satisfy two requirements:
−sin α˜n cos(3α)
cos α>1/n

These will now be derived, based on the angular relationships shown in FIG. 10. The incident angle to the total internal reflection face is (90−α) degrees. The critical angle assuming the cone is in air is:

sin θ_{c}=1/n

Hence, for total internal reflection:

(90−α)>θ_{c}

sin(90−α)>1/n so that cos α>1/n

The reflected light has an angle of incidence on the second face (with respect to the normal) of 3α−90 degrees. This is shown diagrammatically in FIG. 10. The exit angle needs to be angle α for the exit light to be perpendicular to the angle of incidence.

Using Snell's law and with n=1 for the air around the cone, this gives:

sin(3α−90)/sin α=1/n

−cos 3α/sin α=1/n

This gives:

−sin α=n cos(3α).

One example for a cone made of a plastics with n=1.49 is α=38.2 degrees. For example PMMA has a refractive index of 1.49. Other transparent polymers or glasses may be used, with typical refractive indices in the range 1.3 to 1.6.

The exit light does not have to be perfectly radial. Different exit angles can achieved by deviating from −sin α=n cos(3α). For example, it may be suitable to select values of n and α which satisfy:

−0.9 sin α/cos(3α)<n<−1.1 sin α/cos(3α)

This also assumes the cone is in air. There may be a coating on the conical face, which will changes the refractive index difference at the exit face, and this can also change the required relationship between angle and refractive index (in that the value n becomes a refractive index ratio at the boundary). The coating may for example have a cylindrical outer shape as shown by dotted lines 823 so that there is no refraction at the exit, but the angular change at the interface between the cone and the coating is altered.

The imaging device such as the camera has in some embodiments a field of view≧90° in order to reduce the geometric length of the sensor. As described herein the light pattern generating element (the GRIN lens configured to collimate the light from an optical fiber and direct the light beam onto the tip of a reflective cone) generates a ring pattern around the catheter. Furthermore the second example comprises the imaging device (the camera), the reflective cone and GRIN lens being fixed along the same optical axis (in contrast with the first example where the imaging device is offset from the optical axis of the light pattern generating element.

It would be understood that the distance between the imaging device (camera) and the reflective cone is determined so that the light pattern projected onto the airway wall is within the field of view of the camera for typical airway sizes. For example the distance of the camera to the cone is typically between 2 cm and 1 cm.

The glass tube or transparent capillary is in some embodiments connected on both sides to the (opaque) flexible catheter parts. Within these flexible catheter parts the cables or connections carrying the image data generated by the imaging device (the camera) and the optical fiber can be contained. The optical fiber is in some embodiments coupled or connected to a laser diode configured to generate visible light, while the ‘camera cable’ is in some embodiments configured to couple the imaging device (camera) and the data processing unit 401.

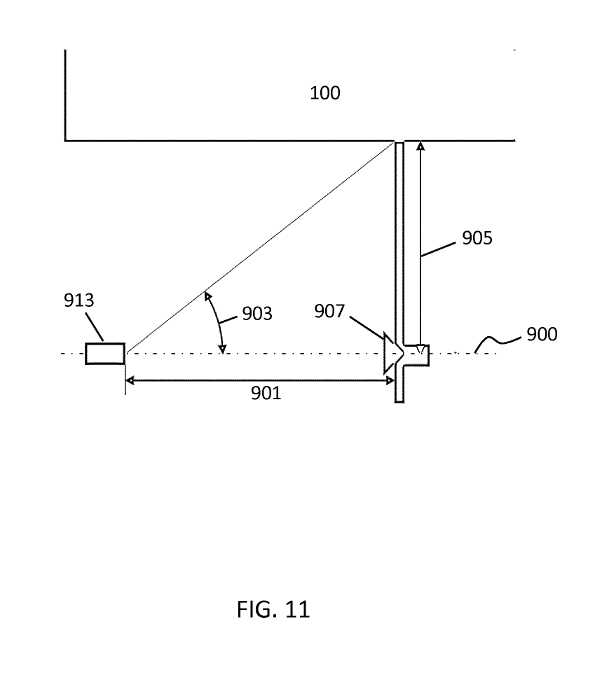

The images received by the data processing unit 401 in some embodiments are first processed to determine whether there are any static and/or dynamic reflection patterns which interfere with or obscure the ring pattern projected onto the airway wall. The data processing unit 401 can then in some embodiments subtract the determined static and/or dynamic reflection patterns to produce a clearer ring pattern. The data processing unit 401 can then in some embodiments track the light pattern on the image and determine the angle with the optical axis at points of the ring patterns as it appears on the image. The data processing unit 401 can then in some embodiments determine from basic geometry the distance to the airway wall. For example as shown in FIG. 11, where the camera 913 is on the optical axis 900 at a defined distance 901 between the camera (imaging device) 913 and the cone (ring pattern generator) 907 and a determined ring angle (camera angle) 903 the airway wall distance 905 from the optical axis may be determined according to the expression:

Airway wall distance=Distance camera-cone*tan (camera angle).

The airway wall distance is thus obtained by triangulation.

It would be understood that in some embodiments the airway wall distance may be determined according to any suitable manner. For example in some embodiments the airway wall distance can be determined by the number of imaging pixels between the ring and the camera or imaging device's optical center for a camera with a fixed field of view and zoom setting.

The data processing unit 401 can in some embodiments determine the ring angle (camera angle) and therefore the airway wall distance for the whole image ring. In some embodiments the determination of the ring angle (camera angle) and therefore the airway wall distance is performed by sampling the image sector by sector and interpolating between sector based airway wall distance calculations. In some embodiments, for example where some parts of the airway are be obscured or in shadow, for example from a camera cable or optical fiber for a more distal sensor passing down through the sensor, then the data processing unit can in some embodiments interpolate the missing part data.

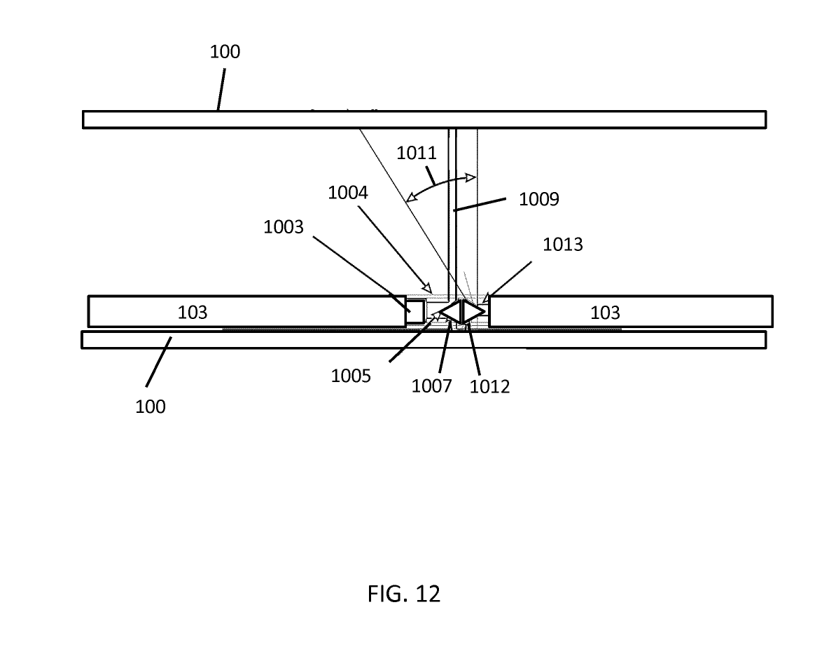

With respect to FIG. 12 a cross sectional view of a third example optical sensor is shown. The third example optical sensor differs from the first two example optical sensors in that the imaging device or camera 811 is coupled to a second reflective element configured to convert the ‘forward looking sensor’ into a ‘sideways looking sensor’.

This design thus has first and second reflective elements which are back-to back with their reflecting surfaces facing outwardly.

The use of two reflective elements enables triangulation to be used to measure the radial distance. The axial spacing between the two reflectors is the base of a triangle. The angle from which light is received into the camera (as determined by the location of the received light within the field of view) can then be combined with the base of the triangle to derive the radial distance in the manner explained above (i.e. airway wall distance=cone-cone distance*tan (angle of incidence).

The ‘sideways looking sensor’ enables the sensor to measure a larger maximum airway wall distance without requiring a longer sensor. As can be shown from the expression (Airway wall distance=Distance camera-cone*tan (camera angle)) a maximum airway wall distance is fixed based on the imaging device's maximum field of view extent and the distance between the camera and cone, therefore to increase the maximum airway wall distance which can be measured for any single camera then either the camera-cone distance or the field of view of the camera is required to be increased. Also as in complicatedly formed airways the ring pattern can be obscured or shadowed by airway structures closer to the camera by employing in some embodiments a second reflective element (which in the example shown in FIG. 12 is cone-shaped) the near ‘shadow’ effect in the forward looking sensors can be improved upon.

The optical sensor as shown in FIG. 12 is located within a transparent capillary or glass tube 1004 within the catheter 103. Furthermore the optical sensor comprises an optical fiber which is configured to transmit a light beam to a gradient index (GRIN) lens 1003 located at the fiber end. The GRIN lens 1003 (or any other suitable lens configuration) is configured to produce a light beam (laser beam) 1105 which is projected within (and generally in the direction along) the glass tube 1004 onto a surface of a reflective cone 1007 also within the glass tube 1004. The reflective cone 1007 is configured to reflect the light such that it passes through the glass tube 1004 wall and generates a ring pattern 1009 which projected onto the airway wall 100. An imaging means or device such as a small camera 1013 which is shown in FIG. 12 located within the glass tube 1004 and is aligned substantially in the opposite direction as the path of the light beam is configured with a field of view 1111 defined by the second reflective cone 1012 and enabled to capture an image comprising the projection of the ring pattern 1009 on the airway wall 100. The image generated by the imaging device or camera 1013 can be furthermore configured to pass the image data to the data processing unit 401.

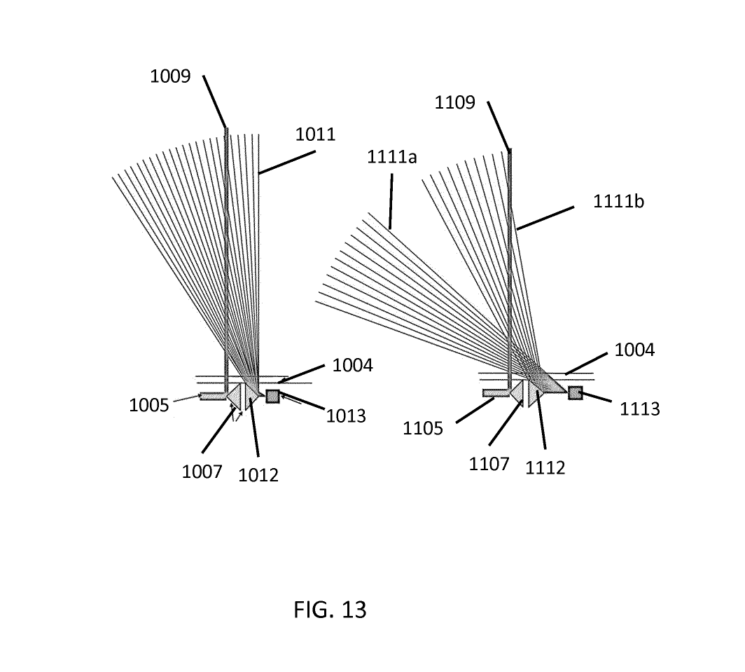

With respect to FIG. 13 two versions of ‘sideway-looking’ optical sensor configurations are shown. The left hand side ‘sideway-looking’ optical sensor configuration (such as implemented within the optical sensor as shown in FIG. 12) has the distance between the imaging device or camera 1013 and the second reflective element or reflective cone 1012 set such that (substantially) the full field of view 1011 of the camera is deflected to the sides. In contrast, in the ‘hybrid’ sensor as shown on the right hand side of the Figure, the second reflective element or reflective cone 1112 is placed at a distance from the imaging device or camera 1113 so that part of the camera field of view is deflected to the sides 1111b but a substantial non-deflected part 1111a remains.

The ‘hybrid’ sensor in some embodiments has certain advantages over both the ‘forward facing sensor’ and the ‘sideways facing sensor configurations’. The light beam 1105 deflected by the first reflective element of reflective cone 1107 generates the ring pattern 1109 on the airway way. The deflected field of view as described herein can be used to detect there the airway wall is relatively far from the sensor and the non-deflected field of view can be configured to detect when the airway wall is very close to the sensor.

Furthermore in some embodiments the configuration of the imaging device (camera 1113) and the second reflective element or reflective cone 1112 can be arranged so that there is an overlap in coverage between the reflected FOV and the direct FOV regions. This overlap in coverage can be configured for the corners of the camera image where the FOV of the camera is largest. In such embodiments where the airway wall falls within this overlap part of the ring is seen twice on the camera, once with respect to the deflected FOV and again in the direct FOV. In some embodiments this ‘double’ ring determination can be used as an error indicator to detect when the sensor is too covered or obscured by secretions to work properly.

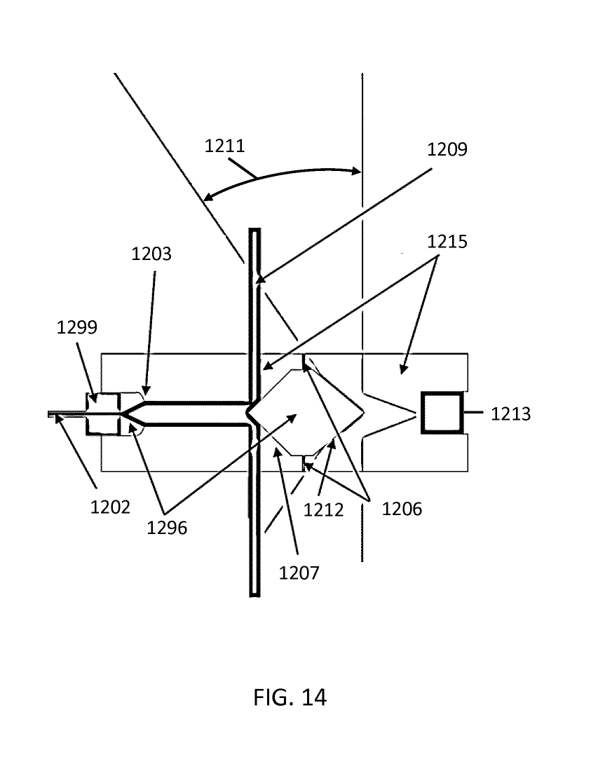

With respect to FIG. 14, a cross sectional view of a fourth example optical sensor is shown. The fourth example optical sensor differs from the third example optical sensor in that the lensing element and reflective elements are generated from air gaps within a molded plastic (or other machined transparent material) rod rather than using GRIN lenses and reflective elements inserted into a glass tube or transparent capillary. The fourth example optical sensor is therefore cheaper and easier to manufacture, needing fewer components to be aligned perfectly. Furthermore in the embodiments implanting such sensors the illumination or light beam passes through fewer interfaces and therefore produces fewer parasitic reflections. For example in the examples shown with respect to FIGS. 8, 9 and 12, the light passes two air-glass (or air-plastic) interfaces (the capillary walls) which create a multitude of reflections and can make it difficult to identify the real ring pattern on the camera image (especially if the presence of secretions creates further interfaces with their reflections).

Thus as shown in FIG. 14 by employing a solid glass or plastic rod into which the cones and or the pattern generating elements are molded, machined or cut. A ‘solid sensor’ can be defined. The reflective elements of both the pattern generator and the imaging system are defined by this solid body having an internal cavity which defines the two cone faces, the internal cavity having a lower refractive index than the material of the solid body.

The ‘solid’ sensor as shown in FIG. 14 comprises two elements which are molded, machined or cut glass or plastic rods 1215 butted together as shown by joins 1206. The first glass or plastic rod comprises two air-filled cavities or hollows 1296, each of which defines an optical component. The first hollow defines a first lens shape 1203 and is configured to receive the fiber 1202 via a fiber pigtail 1299. The second hollow defines a first reflective element or cone shape 1207, configured to reflect the light beam to generate the ring pattern 1209 projected onto the airway wall. The second glass or plastic rod comprises a second reflective element or cone shape hollow 1212 for reflecting the imaging device (or camera 1213) field of view. In some embodiments if the refractive index of the rod and the cone angle are suitable, then total internal reflection occurs at the cone-air boundary and the cone-shaped cut functions like a reflective cone. Alternatively in some embodiments an additional reflection layer is applied to produce the reflective surface.

The imaging device or camera 1213 can be located or aligned within the sensor by the second glass or plastic rod having a imaging device or camera bore, hollow or hole within which the camera is fitted. As such the imaging device is configured with a field of view 1211 defined by the a second reflective element or cone shape hollow 1212 to capture an image comprising the projection of the ring pattern 1209 on the airway wall. The image generated by the imaging device or camera 1213 can be furthermore configured to pass the image data to the data processing unit 401.

Furthermore although the example shown with respect to FIG. 14 shows a two-piece or two-element ‘solid’ sensor part or portion it would be understood that in some embodiments the ‘solid’ sensor is formed from any suitable number of pieces or elements.

In some embodiments the solid sensor as discussed herein comprises of two solid rods of transparent glass or plastic which are glued together, using any suitable glue, at joins 1206.

In order to guide cables and fibers to further sensors in some embodiments the rods are scored or grooved.

Although the example solid sensor as shown in FIG. 14 is a sideways looking sensor it would be understood that a forwards looking sensor could also be implemented in a solid senor configuration.

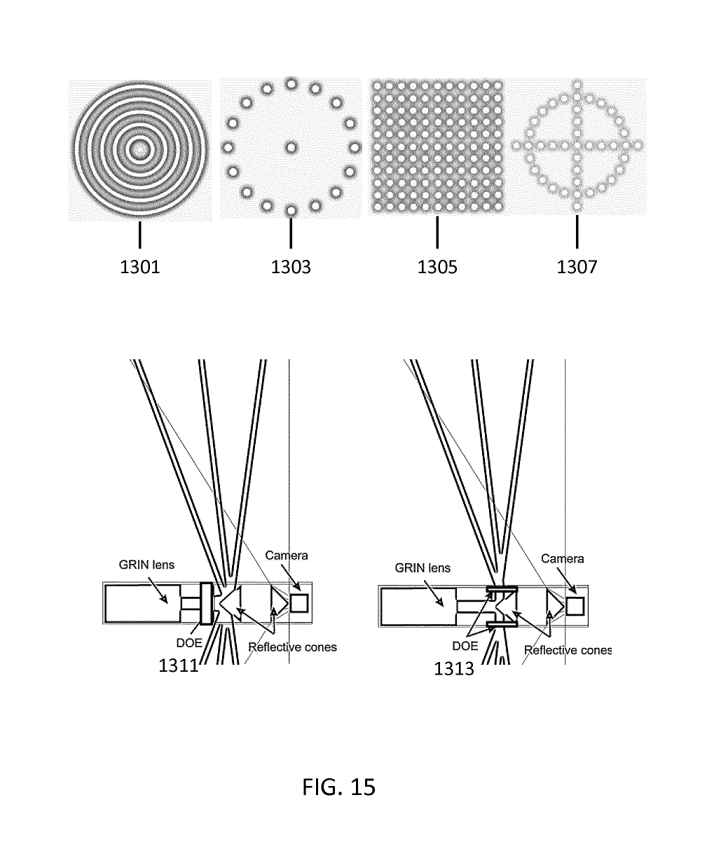

In the examples as discussed herein the light ring projected onto the airway wall is a substantially whole ring. However in some embodiments the light pattern projected can be more sophisticated than a simple ring. In such embodiments the light pattern can provide a better or more robust reconstruction of the airway. Thus in some embodiments a light pattern is generated by implementing or employing a Diffractive optical element (DOEs) plate or foil. A custom made DOEs can in such embodiments produce nearly arbitrary diffraction patterns in the transmitted beam. Some examples are shown in FIG. 15 as shown by the plate of foil patterns 1301, 1303, 1305, and 1307.

Furthermore it would be understood that the implementation of a DOE can be performed in any suitable way. For example as shown in the left hand side of FIG. 15 a sensor can be configured with a DOE foil or grid 1311 located between the GRIN lens and the reflective cone. Alternatively in some embodiments a DOE foil or grid 1313 can be located around the cone reflective cone, such as shown in the right hand side of FIG. 15. The DOE shown in this example creates 3 concentric rings. The additional rings can in such embodiments be used to improve the precision with which the distance to airway walls can be measured. Although the examples shown in FIG. 15 show a DOE foil or grid implemented within a sideways looking sensor it would be understood that a DOE foil or grid could furthermore be incorporated within the ‘forwards looking sensor’ or ‘solid sensor’ examples as discussed herein.

In some embodiments a holographic element (for example a holographic crystal or a holographic polymer element) can be employed to creates a holographic (or complex) light pattern when illuminated.

In the examples as discussed herein the reflective elements (or reflective cones) were described as straight cones, in other words the reflective surface is linear. In some embodiments however the reflective surface can be non-straight (or in other words discontinuous or non-linear) in order to integrate additional functionality.

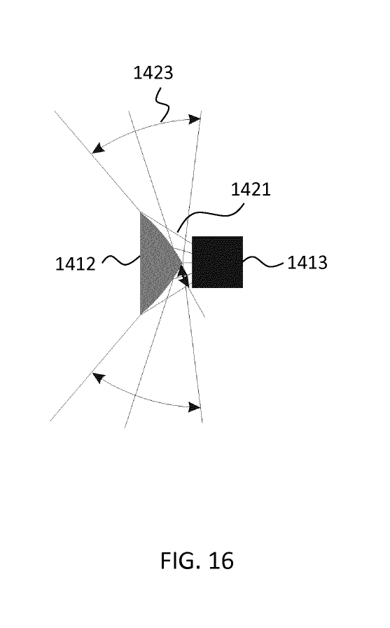

For example with respect to FIG. 16 is shown a sensor comprising a reflective cone 1412 to reflect the imaging device or camera 1413 field of view which is configured to have a curved reflective surface. It would be understood that the field of view 1423 of the sensor is usually limited to the field of view 1421 of the camera, however by employing a reflective cone with concave side, it is possible to increase the sensor field of view and produce the same effect as adding a concave lens, but without the additional cost and alignment issue. The concave reflective cone shape can be implemented with respect to the reflective cones according to ‘forwards looking sensor’, ‘sideways looking sensor’ or either implementations of the ‘solid sensor’ embodiments. Implementing a concave cone to increase the field of view is especially advantageous in a sensor in a ‘forwards looking sensor’ embodiments where the beam reflective cone surface is concave is because with a larger field of view it becomes possible to build a shorter sensor without compromising the maximum airway wall distance that can be measured.

In some embodiments it would be understood that a curved cone could be implemented in order to replace the GRIN (or other) lens for generating the beam for providing the projected ring.

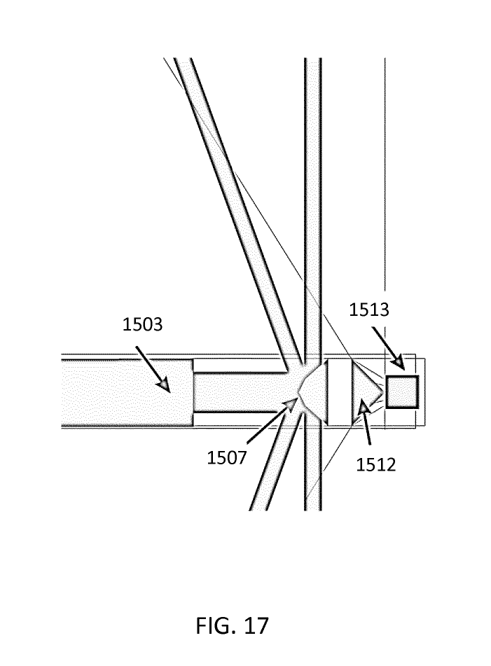

In some embodiments the reflective elements or reflective cones can comprise step-wise straight sides but with different angles. For example by implementing a step-wise reflective surface with different angles as the light beam reflective element a more sophisticated light pattern can be generated, rather than a simple cone. For example in some embodiments such as shown in FIG. 17 the light from the GRIN lens 1503 is reflected by the stepped cone with two angles 1507 to produce a light pattern of two rings. Reflective cone 1512 is correspondingly chosen having appropriate geometric properties to provide a projected camera 1513 field of view wide enough to capture all generated ring patterns.

Although a single step change producing a cone with two reflective angles is shown it would be understood that more than one step change can be implemented in some embodiments. Furthermore it would be understood that in some embodiments by implementing a convex step change on the imaging device reflective element then a coverage overlap area can be created enabling an error detection operation as discussed herein.

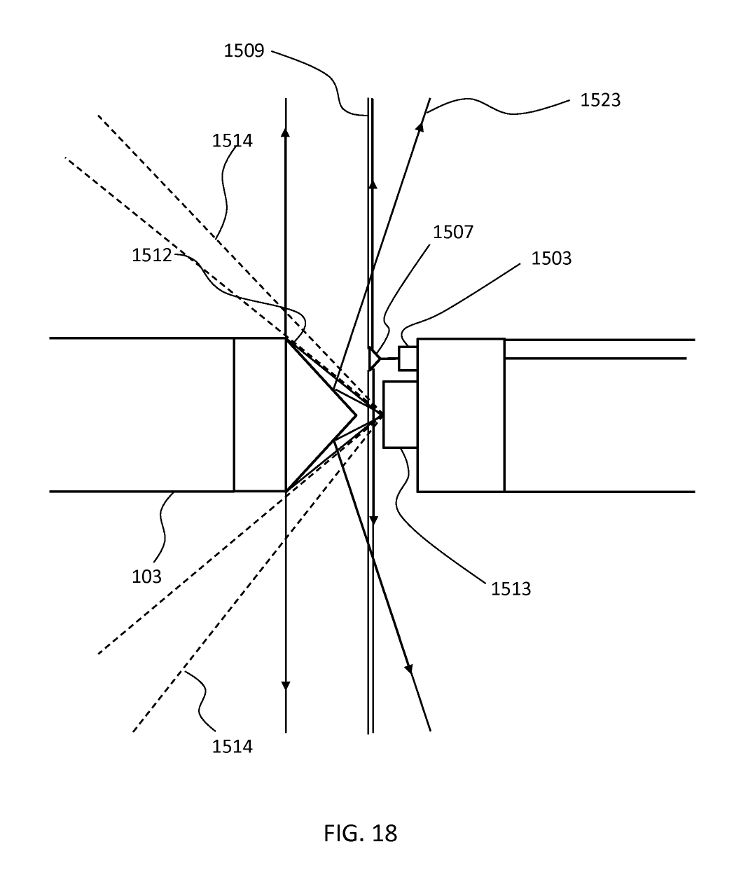

According to another example embodiment of the catheter sensor, illustrated in FIG. 18, the camera 1513 and GRIN lens 1503 are arranged in the same radial plane, offset from each other. The camera has a field of view with a central axis aligned with the catheter central axis, and the camera cone 1512 is accordingly also centered on the catheter central axis. The light dispersing cone 1507 also received incident light parallel to the catheter elongate axis, but offset from the central axis. The camera cone 1512 and the light dispersing cone face in the same longitudinal direction. As can be seen by FIG. 18, this is achieved by employing a light dispersing cone 1507 of a size very much smaller than the respective camera cone 1512, and by mounting said light dispersing cone at a point radially displaced from the center of the catheter, outside of the field of view of the camera 1513.

Light is propagated from GRIN lens 1503 toward the surface of the light reflecting cone 1507. The light reflecting cone has surface adapted to reflect all incident light such that it exits the catheter at a perpendicular (i.e. radial) angle with respect to the catheter longitudinal axis. The reflected radial beam projects ring pattern 1509 at the surrounding airway walls.

Since the light dispersing cone 1507 reflects light perpendicularly, its off-center position has no effect on the radial symmetry of the generated ring pattern 1509. This would be in contrast, for example, to a cone adapted to reflect light at a range of angles, wherein an off-center positioning would generate a ring pattern having differing widths at different points about the circumference of the airway.

As shown by light path 1514, the camera 1513 (as an optional feature) has a field of view which is wide enough to capture light from further in front. This enables contour mapping using distance measurement based on the ring pattern, and also conventional imaging based on the light 1514. This enables two-way viewing.

The light reflecting cone 1507 may be adapted to produce radial propagation by, for example, employing a conical surface with an inclination of 45 degrees with respect to the longitudinal axis of the catheter, in combination with incident propagation vector from the GRIN lens 1503 substantially parallel with said axis. With this arrangement, light incident from the lens reflects from the 45 degree cone surface at an angle normal to the surface of the catheter (i.e. in a radial direction).

At the same time, camera cone 1512 projects an image of a ringed section of the airway, defined by sensor field of view 1523, and illuminated by ring pattern 1509, into the (horizontal) camera 1513 field of view. Such arrangement is substantially the same as those described in relation to previous embodiments of catheter sensors, depicted by FIGS. 12-17.

The generated sensor field of view 1523 is ideally wide enough to encompass ring pattern 1509, while narrow enough to exclude capturing light dispersing cone 1507. The camera cone 1512 in some examples of this embodiment may have surface inclination angle at the apex of approximately 126 degrees. At this angle, the cone is for example able to project images to the camera 1513 from surfaces which are at a radial distance of between 0.32 and 29.7 mm from the outer perimeter of the catheter 103. This is based on a catheter outer diameter of 3 mm, and is the range of radial distances for which the projected field of view 1523 overlaps with the ring pattern 1509, The rigid sensor section of the catheter is this example may have length of approximately 10 mm. Of course, different designs are possible by altering the various dimensions, angles and relative positions.

The light reflecting cone 1507 may be a specular surface-reflecting cone (e.g. aluminum) or it may be a total internal reflecting cone (with the tip facing the opposite direction) as explained above with reference to FIG. 10.

This example shows that the light source and light source reflector do not need to be aligned with the central catheter axis. Similarly, the image sensor and image sensor reflector do not need to be aligned along the central axis. Thus, the imaging system may instead be off center, or indeed both units may be off center. Different designs will give different ways of using the available space.

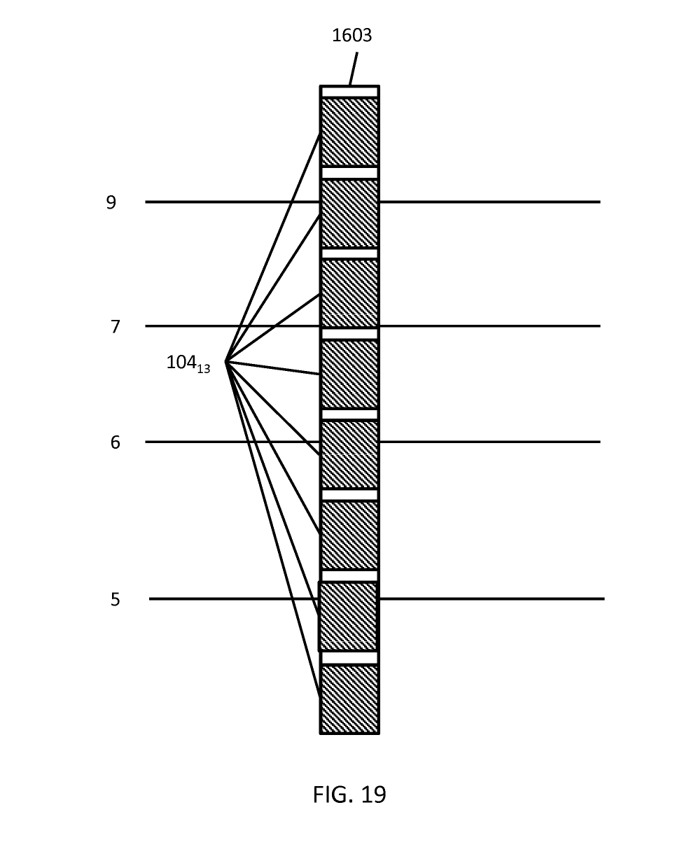

With respect to FIG. 19 a second or further configuration of sensor arrangement on the catheter is shown. In such embodiments rather than, as shown in FIG. 4, locating or placing a limited number of sensors 104 on the upper airway catheter 103 so that the sensors are aligned to regions of special interests such as the velum (V) 9, oropharynx (O) 7, base of tongue (T) 6, and epiglottis (E) 5 the catheter 1603 comprises a multitude of sensors 104₁₃ distributed along the catheter 1603 so that the whole length of interest is covered with sensors 104₁₃. In such embodiments the spacing of the sensors 104₁₃ may be chosen in such a way that the spacing is small enough to get a representative representation of the airway regardless of the size of the airway. Furthermore in such embodiments the data processing unit 401 may be configured to interpolate the cross sections at a given position in the airway which is not directly covered by a sensor 104₁₃ by interpolating neighboring sensor cross sections.

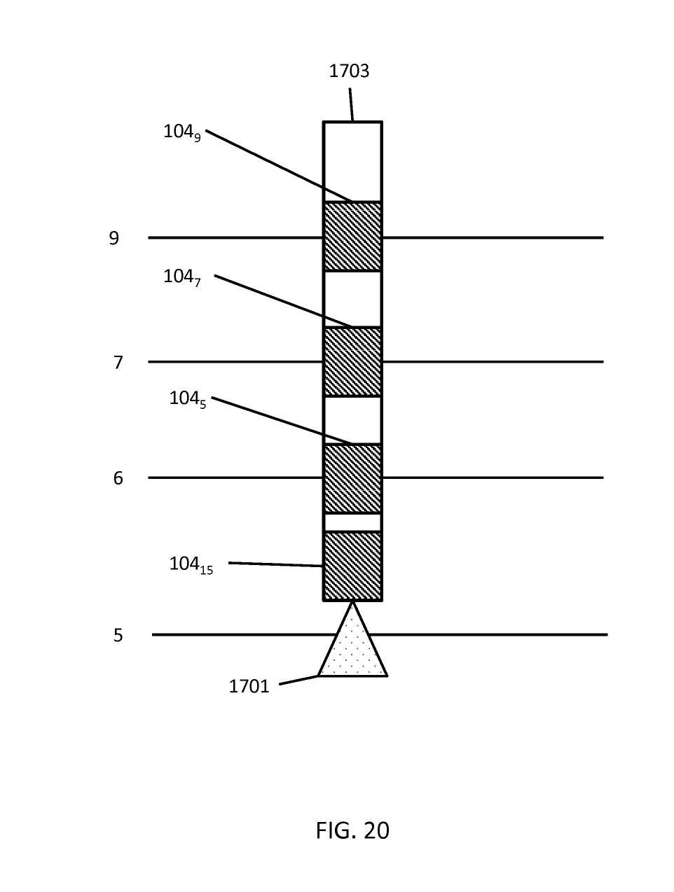

With respect to FIG. 20 a third configuration of sensor arrangement on the catheter is shown.

In such embodiments the catheter 1703 is configured with sensors 104₅, 104₇, 104₉ located to provide airway cross section information in the velum 9, oropharynx 7 and base of tongue 6 regions respectively. However to provide more information about the epiglottis 5 region and furthermore to prevent any gag reflex where the catheter may come in contact with the epiglottis the catheter 1703 comprises a downward looking sensor 104₁₅ in the tip of the catheter that specially monitors the epiglottis 5. The catheter 1703 in such embodiments would therefore be a shorter catheter ending just before the epiglottis 5. In some embodiments the ‘epiglottis’ sensor 104₁₅ may comprise a light pattern generating element with a defined light pattern 1701 and an imaging device, such as a camera for imaging the epiglottis under the light pattern. However it would be understood that in some embodiments the ‘epiglottis’ sensor 104₁₅ may be a 2-dimensional array of ultrasound transducers configured to produce a ‘view’ of the epiglottis from the tip or end of the catheter 1703. In other words the catheter sensor configuration is such that there is in such embodiments at least one sensor located at the distal end of the catheter configured to observe or measure the volume (such as the airway as discussed herein) adjacent the distal end of the catheter as well as at least two sensors distributed spaced along the catheter.

Although the example shown herein with respect to FIG. 20 shows a sensor arrangement or configuration similar to the first example, such as shown in FIG. 4 but with the ‘epiglottis’ sensor it would be understood that the sensor arrangement or configuration may be similar to the second example, such as shown in FIG. 19 with the end sensor replaced with the ‘epiglottis’ sensor.

In the examples shown herein the optical sensor is described with respect to a medical catheter. However it would be understood that in some embodiments the optical sensor can be implemented within and along any suitable sensor array configuration for monitoring cross sectional regions or pipes, conduits of any suitable shape or size. For example the optical sensor can in some embodiments be implemented within a sensor array deployed within a volume for the observation or checking of cross-sectional consistency. Thus where pipeline or pipes are subject to external pressure, which may cause constriction to flow within the pipe, then the optical sensors can provide an indication of the location of any collapse or constriction.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The embodiments may be implemented by means of hardware comprising several distinct elements. In the device claim enumerating several means, several of these means may be embodied by one and the same item of hardware. The mere fact that certain measures are recited in mutually different dependent claims does not indicate that a combination of these measures cannot be used to advantage. Furthermore in the appended claims lists comprising “at least one of: A; B; and C” should be interpreted as (A and/or B) and/or C.

1. An optical sensor for operating along a catheter, the optical sensor having a sensor length along a direction parallel to the catheter direction, the optical sensor comprising:
a light pattern generator configured to project at least one light pattern at a radial projection angle with respect to the optical sensor length onto the inner surface of an elongated volume into which the optical sensor is inserted, wherein the light pattern generator comprises an illumination system for providing a first light beam having a component of its direction along the sensor length and a first light redirection element configured to redirect the light beam to generate the at least one light pattern at an oblique and/or right angle to the optical sensor length;
a second light redirection element for redirecting a reflected version of the light pattern from the inner surface of the elongated volume to provide a second light beam; and
an imaging device having a field of view with a central axis substantially parallel to the length of the optical sensor for receiving the second light beam.

2. An optical sensor as claimed in claim 1, wherein the first light redirection element comprises a first reflective element and the second light redirection element comprises a second reflective element.

3. An optical sensor as claimed in claim 2, wherein the light pattern generator comprises a lens configured to output a light beam to the first reflective element.

4. An optical sensor as claimed in claim 2, wherein the first reflective element comprises a reflective cone.

5. An optical sensor as claimed in claim 4, wherein the reflective cone comprises:
a reflective surface with a single slope angle configured to generate the at least one light pattern in the form of a ring at an oblique and/or right angle to the sensor length; or
a stepped reflective surface having at least two different reflective surface slope angles and configured to generate at least two light patterns in the form of at least two rings at an oblique and/or right angles to the sensor length; or
a varying angle reflective surface angle configured to generate a distributed light pattern at an oblique and/or right angle to the sensor length.

6. An optical sensor as claimed in claim 5, wherein the cone is arranged to reflect light based on total internal reflection.

7. An optical sensor as claimed in claim 6, wherein the first reflective element comprises a solid body having an internal cavity which defines the cone, the internal cavity having a lower refractive index than the material of the solid body.

8. An optical sensor as claimed in claim 7, wherein the internal cavity defines the first and second reflective elements facing in opposite directions.

9. An optical sensor as claimed in claim 1, wherein the first and second light redirection elements are back-to back with light redirecting surfaces facing outwardly.

10. An optical sensor as claimed in claim 1, wherein the first light redirecting element is offset from a central axis of the optical sensor and, during use, the first light beam is offset from the central axis, and the second light redirecting element is centered on the central axis, wherein the first and second light redirecting elements face in the same direction.

11. An optical sensor as claimed in claim 1, wherein the imaging device comprises a camera located within the optical sensor.

12. An optical sensor as claimed in claim 1, wherein the second reflective element comprises at least one of:
a reflective cone comprising a single reflective surface angle configured to reflect at least part of the field of view of the camera from an axial field of view direction to a radial field of view direction;
a reflective cone comprising a stepped reflective profile having at least two different reflective surface angles and configured to reflect a first part of the field of view of the camera from an axial field of view direction to a first range radial field of view directions, and a second range of the field of view of the camera from an axial field of view direction to a second range radial field of view directions non continuous with the first range radial field of view directions;
a reflective cone comprising a varying reflective surface angle and configured to generate a sensor field of view range greater than the field of view of the camera; or
a reflective cone comprising a varying reflective surface angle and configured to generate a sensor field of view range less than the field of view of the camera.

13. The optical sensor as claimed in claim 1, further comprising a transparent capillary configured to support the at least one light pattern generator, the imaging device and the second light redirection element, and further permit the transmission of the at least one light pattern from optical sensor to the inner surface of the elongated volume.

14. A catheter comprising at least two optical sensors each as claimed in claim 1, the at least two optical sensors being distributed spaced along the catheter, and wherein the at least two optical sensors are configured to observe different substantial cross sections of the inner surface of the volume within which the catheter is located.

15. An imaging method for obtaining images from an optical sensor provided along a catheter wherein the sensor is configured to observe a cross section of an elongated volume within which the catheter is located, the optical sensor having a sensor length along a direction parallel to the catheter direction, the method comprising:
projecting at least one light pattern at a radial projection angle with respect to the optical sensor length onto the inner surface of the elongated volume into which the optical sensor is inserted, the projecting comprising providing a first light beam having a component of its direction along the sensor length and using a first light redirection element to redirect the light beam to generate the at least one light pattern at an oblique and/or right angle to the optical sensor length;
redirecting a reflected version of the light pattern from the inner surface of the elongated volume using a second light redirection element to provide a second light beam; and
receiving the second light beam using an imaging device having a field of view with a central axis substantially parallel to the length of the optical sensor.
